(19) United States
(12) Patent Application Publication
(10) Pub. No.: US 2007/0032868 A1
(43) Pub. Date: Feb. 8, 2007

(54) Capsular shape-restoring device

(76) Inventor: Randall Woods, Gilbert, AZ (US)

Correspondence Address: HOVEY WILLIAMS LLP, 2405 GRAND BLVD., SUITE 400, KANSAS CITY, MO, 64108, US

(21) Appl. No.: 11/199,395

(22) Filed: Aug. 8, 2005

(51) Int. Cl. A61F 2/16

(52) U.S. Cl. 623/6.39

(57) ABSTRACT

A capsular shape-restoring device (28) and a method of using that device (28) are provided. The device (28) is designed for surgical implantation within the capsule (20) of an eye (10) and includes a body (42) and optionally an optic (62). The body (42) is discoid in shape and generally conforms to the shape of the natural capsule (20). As a result, the device (28) assists in returning the capsule (20) to its natural shape as it exists prior to removal of the natural crystalline lens. The device (28) does not provide accommodation, even when coupled with an optic (62).

### BACKGROUND OF THE INVENTION

1. Field of the Invention

The present invention relates to non-accommodating, capsular shape-retaining devices that can be surgically implanted to restore the capsular bag to its natural shape after removal of the natural crystalline lens.

2. Description of the Prior Art

There are often situations that require the removal of the natural crystalline lens from the capsule or capsular bag (e.g., cataracts). Cataracts occur when the crystalline lens of the eye becomes opaque. The cataracts may be in one or both eyes and, being a progressive condition, will cause fading vision and eventually blindness in most cases. Cataracts are typically surgically removed along with the natural crystalline lens and the anterior wall of the capsule of the eye. In the past, an artificial lens was implanted so that vision could be achieved at either near distance or far distance, with corrective lenses being required for viewing at the other distance. Presently, improvements are being made in artificial lenses to provide at least some accommodation. Many of these lenses are implanted outside the capsular bag (e.g., anterior to the bag), while some accommodative lenses are implanted within the capsular bag. However, even those that have been implanted within the capsule thus far are flat and do not conform to the natural shape of the capsular bag.

If the shape of the bag is not restored to its natural shape after removal of the natural lens, many problems can occur. For example, even though the natural lens has been removed, lens cells will remain, and the eye will often attempt to regrow a lens from those cells. If this new growth is located on the posterior wall of the capsule, the patient will have posterior capsule opacity (i.e., secondary cataract) that must be treated. These new cells cannot be surgically removed but instead must be dislodged with laser treatment. However, in a certain percentage of these cases, the laser treatment will cause debris to travel to the retina, thus causing cystoid macular edema.

Another problem that can occur after removal of the natural lens is that the solid volume in the eye has been decreased, and the vitreous behind the eye has more freedom to move. The vitreous is attached to the retina in some locations, and this increased movement may pull on the retina, thus causing retinal detachment. The risk of retinal detachment is even greater if the patient was nearsighted prior to cataract removal.

There is a need for a device that can return the capsule to its natural shape and prevent the above problems. This device should be readily insertable into the capsule and should last for a substantial number of years without damaging any of the eye components.

### SUMMARY OF THE INVENTION

The present invention fills this need by providing non-accommodative, intraocular, capsular, shape-restoring devices that are safe for long-term use in the eye.

In more detail, the shape-restoring devices of the invention comprise a positioning body, optionally coupled to an optic, that presents a discoid-shaped device generally conforming to the shape of the natural eye capsule. The positioning body is preferably unitarily formed, and presents a posterior face that engages the posterior wall of the natural capsule, and an anterior face that engages the anterior wall of the natural capsule. The anterior and posterior faces of the positioning body are joined together by a curved sidewall that optionally includes openings formed therein.

There is an opening formed in at least one of the posterior face or anterior face. In some embodiments, the other of the anterior and posterior faces will include an optic, while in other embodiments, there is an opening at both faces.

### BRIEF DESCRIPTION OF THE DRAWING FIGURES

FIG. 1 is a vertical sectional view showing placement of a capsular shape-restoring device of the invention within the capsule of an eye;

FIG. 2 is a side elevational view of the device of FIG. 1;

FIG. 3 is a vertical sectional view showing placement of an alternative embodiment of the inventive capsular shape-restoring device similar to FIG. 1, but with an optic;

FIG. 4 is a side elevational view of the device of FIG. 3;

FIG. 5 is a perspective view of an alternative embodiment of the inventive capsular shape-restoring device;

FIG. 6 is a perspective view of an alternative embodiment of the inventive capsular shape-restoring device similar to the embodiment of FIG. 5, but with an optic;

FIG. 7 is a perspective view of an alternative embodiment of the inventive capsular shape-restoring device;

FIG. 8 is a section through a perspective view of an alternative embodiment of the inventive capsular shape-restoring device;

FIG. 9 is an exploded perspective view of the device of FIG. 8; and

FIG. 10 is a perspective view of an alternative embodiment of the inventive capsular shape-restoring device.

### DETAILED DESCRIPTION OF THE PREFERRED EMBODIMENTS

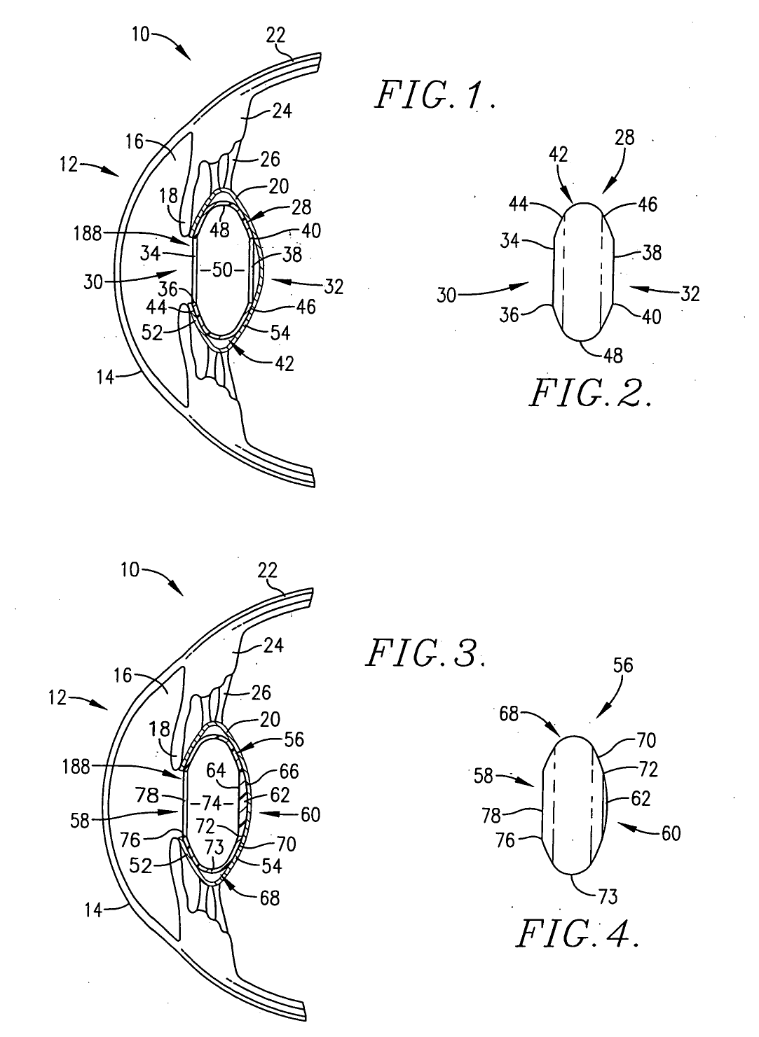

Referring now to the drawings, FIG. 1 shows the various components of the human eye pertinent to this invention. Briefly, the eye 10 includes a frontal portion 12 and a rearward portion (not shown). The frontal portion 12 of the eye 10 is covered by a cornea 14 that encloses and forms an anterior chamber 16. The anterior chamber 16 contains aqueous fluid and is bounded at the rear by an iris 18. The iris 18 opens and closes to admit appropriate quantities of light into the inner portions of the eye 10. The eye 10 includes a capsule 20 (“capsule” and “capsular bag” are used interchangeably herein) that ordinarily contains the natural crystalline lens. When the eye 10 focuses, the capsule 20 changes shape to appropriately distribute the light admitted through the cornea 14 and the iris 18 to the retina (not shown) at the rearward portion of the eye 10.

The retina is composed of rods and cones which act as light receptors. The retina includes a fovea, which is a rodless portion that provides for acute vision. The outside of the rearward or posterior portion of the eye 10 is known as the sclera 22, which joins into and forms a portion of the covering for the optic nerve. Images received by the retina are transmitted through the optic nerve to the brain. The area between the retina and the capsule 20 is occupied by vitreous fluid. Finally, the eye 10 includes a ciliary muscle or body 24 having zonular fibers 26 (also referred to as zonules) that are attached to the capsule 20.

Ocular adjustments for sharp focusing of objects viewed at different distances are accomplished by the action of the ciliary body 24 on the capsule 20 and natural crystalline lens (removed in the accompanying figures) through the zonular fibers 26. The ciliary body 24 contracts, allowing the capsule 20 to return to a more spherical shape for viewing objects that are nearer the viewer. When the ciliary body 24 retracts and pulls on the zonular fibers 26 to make the capsule 20 more discoid, objects at a distance can be viewed in proper focus.

### 1. Embodiment of FIGS. 1 and 2

Referring to FIGS. 1-2, a capsular retaining device 28 is shown. Device 28 comprises an anterior portion 30 and a posterior portion 32. Anterior portion 30 includes an anterior opening 34 formed by anterior perimeter 36, while posterior portion 32 includes a posterior opening 38 formed by posterior perimeter 40.

The device 28 further includes a positioning body 42. Body 42 comprises an anterior outer wall 44, which extends radially from anterior perimeter 36 and a posterior outer wall 46, which extends radially from posterior perimeter 40. Walls 44 and 46 then converge and form a curved sidewall 48. Walls 44 and 46 cooperate to form a chamber 50. Openings 34 and 38 communicate with the chamber 50 allowing fluids to enter and fill the chamber 50.

The overall shape of device 28 generally conforms to the shape of capsule 20. That is, the outer walls 44 and 46 cooperate to form a device having an overall discoid or saucer-like shape as best shown in FIG. 2. The device 28 is of sufficient size so that anterior outer wall 44 of device 28 mildly urges against anterior wall 52 of the capsule 20, while posterior outer wall 46 of device 28 mildly urges against the posterior wall 54 of the capsule 20.

### 2. Embodiment of FIGS. 3 and 4

FIGS. 3 and 4 show an alternative embodiment of the present invention. That is, a capsular retaining device 56 is illustrated. Like device 28, device 56 comprises an anterior portion 58 and a posterior portion 60. However, unlike device 28, device 56 comprises an optic 62 at posterior portion 60. The optic 62 comprises an anterior surface 64 and a posterior surface 66. The anterior surface 64 and the posterior surface 66 usually cooperate to form an optic that is plano-convex (shown) or bi-convex in cross-section, although the shape of these surfaces and size of the optic 62 can be varied depending upon the user's eyesight.

The device 56 further includes a positioning body 68 comprising an outer wall 70 that extends radially from optic 62. In this embodiment, body 68 is preferably integral and essentially flush with optic 62 at optic perimeter 72 where wall 70 joins optic 62. Wall 70 then curves to form a curved sidewall 73, and converges on the anterior portion 58 of device 56. Wall 70 forms a chamber 74 and terminates at location 76 to form an opening 78 that communicates with the chamber 74 allowing fluids to enter and fill the chamber 74.

As with device 28, the overall shape of device 56 generally conforms to the shape of the capsule 20 with outer wall 70 of the body 68 cooperating with optic 62 to form a capsular shape-retaining device having an overall discoid or saucer-like shape as best shown in FIG. 4. The device 56 is of sufficient size so that optic 62 mildly urges against the posterior wall 54 of the capsule 20, while the posterior portion 60 of device 56 urges against the anterior wall 52 of the capsule 20.

The embodiment of FIGS. 3-4 has been described as a capsular-shape retaining device having an anterior optic 62 and a posterior opening 78, but it will be appreciated that the device 56 can be reversed within the eye. That is, it could be positioned to have a posterior opening and an anterior optic. Either way, the device 56 does not accommodate.

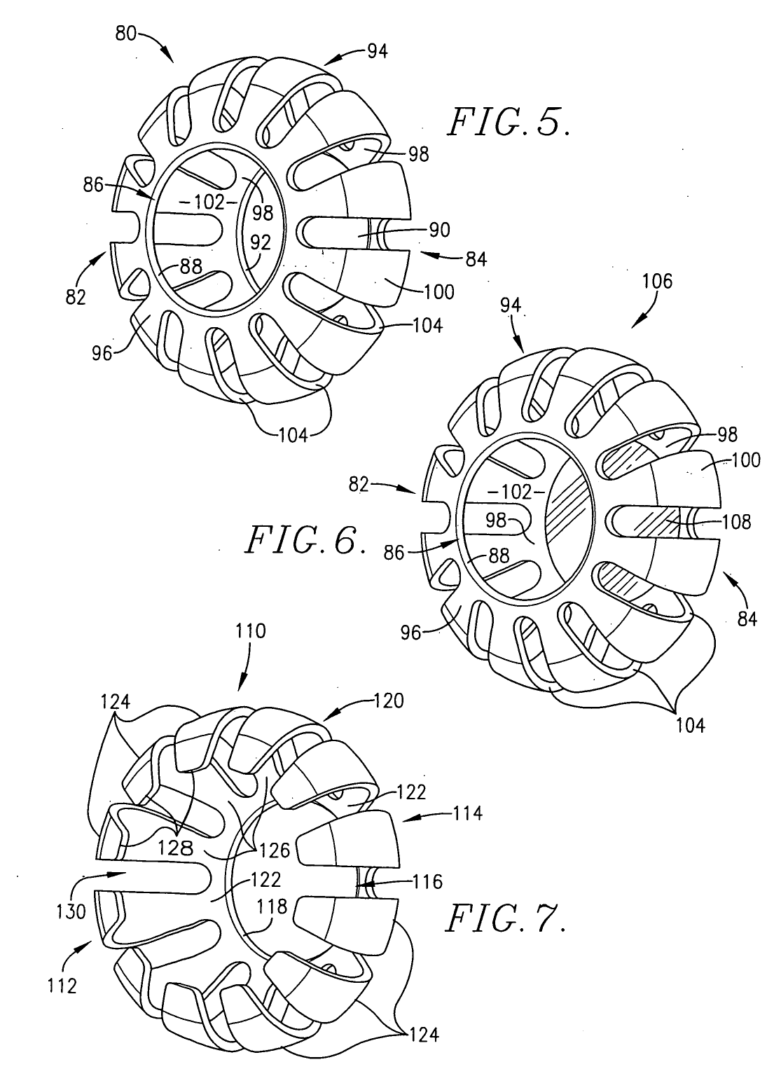

### 3. Embodiment of FIG. 5

FIG. 5 depicts another embodiment of the inventive capsular shape-retaining device. In this embodiment, shape-retaining device 80 is similar to device 28 of FIGS. 1-2 in that it comprises an anterior portion 82 and a posterior portion 84. Anterior portion 82 includes an anterior opening 86 formed by anterior perimeter 88, while posterior portion 84 includes a posterior opening 90 formed by a posterior perimeter 92.

The device 80 further includes a positioning body 94 comprising an anterior outer wall 96 that extends radially from anterior perimeter 88 and a posterior outer wall 98 that extends radially from posterior perimeter 92. Walls 96 and 98 converge and form a curved sidewall 100 and a chamber 102. Curved sidewalls 100 comprise a plurality of sidewall openings 104. Sidewall openings 104 and openings 86 and 90 communicate with the chamber 102, allowing fluids to enter and fill the chamber 102.

### 4. Embodiment of FIG. 6

FIG. 6 depicts an embodiment similar to that of FIG. 5, and like numbers are used to designate like parts. In FIG. 6, capsular shape-retaining device 106 differs from device 80 in that device 106 includes an optic 108 at posterior portion 84. As was the case with the optic 62 of FIGS. 3-4, the shape and size of the optic 108 can be varied depending upon the user's eyesight. Also, the device 106 can be reversed within the eye so that it would have a posterior opening and an anterior optic. Again, the device 106 does not provide accommodation.

### 5. Embodiment of FIG. 7

Referring to FIG. 7, a capsular shape-restoring device 110 is shown. Device 110 comprises an anterior portion 112 and a posterior portion 114. Posterior portion 114 includes a posterior opening 116 formed by posterior perimeter 118.

The device 110 further includes a positioning body 120. Body 120 comprises a posterior wall 122, and posterior wall 122 comprises a plurality of spaced-apart legs 124 radiating therefrom. The legs 124 are arcuate in cross-section and comprise respective base ends 126 and respective free ends 128. As illustrated, base ends 126 are integrally formed with posterior wall 122, while free ends 128 terminate without contacting another part of the device 110, forming an anterior opening 130.

As with the previously described embodiments, the overall shape of device 110 generally conforms to the shape of capsule 20. Also, while this embodiment has been depicted without an optic, it will be appreciated that an optic of the desired size and shape can be included at either anterior opening 130 or posterior opening 116. If the optic is positioned at anterior opening 130, obviously legs 124 will no longer comprise free ends, and the device 110 will resemble the embodiment of FIG. 6 except that the ends 128 of the legs 124 will contact the optic without being connected to one another directly.

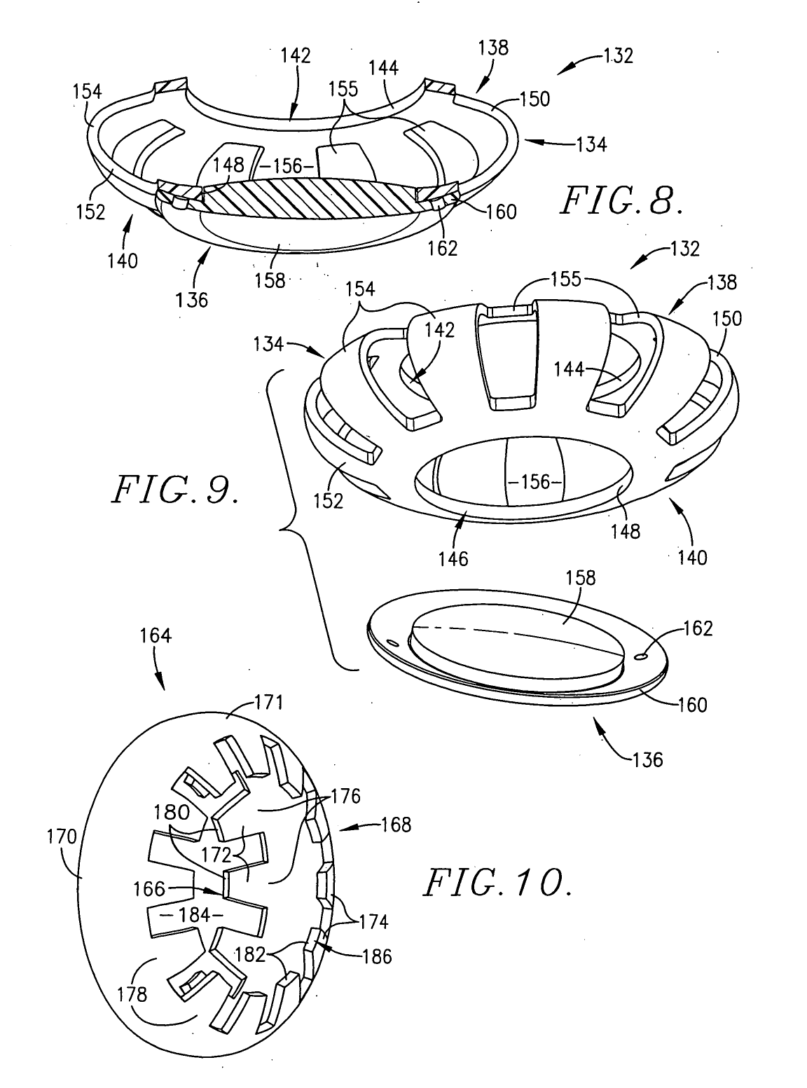

### 6. Embodiment of FIGS. 8-9

It will be appreciated that each of the foregoing embodiments that are depicted with an optic could be designed to have an optic joined to the device as a 2-piece unit rather than an optic that is integrally formed with the device body. One example of such a device is capsular shape-retaining unit 132 of FIGS. 8-9.

Unit 132 comprises a positioning body 134 and an optic combination 136. Body 134 comprises an anterior portion 138 and a posterior portion 140. Anterior portion 138 includes an anterior opening 142 defined by anterior perimeter 144, while posterior portion 140 includes a posterior opening 146 defined by posterior perimeter 148.

Body 134 further includes an anterior outer wall 150, which extends radially from anterior perimeter 144 and a posterior outerwall 152, which extends radially from posterior perimeter 148. Walls 150 and 152 converge and form a curved sidewall 154, having a plurality of spaced-apart openings 155. Walls 150 and 152 cooperate to form a chamber 156. Openings 142 and 146 communicate with the chamber 156 allowing fluids to enter and fill the chamber 156.

Optic combination 136 comprises a bi-convex optic 158 and an annular flange 160 extending radially from the optic 158. Annular flange 160 optionally includes positioning holes 162. The optic combination 136 is preferably attached to the positioning body 134 prior to insertion within the capsule 20, although there may be situations where it is desirable to attach it to the positioning body 134 after insertion in the capsule 20. The optic combination 136 is preferably attached to the posterior opening 146 of body 134 so that the optic is in the posterior position.

As illustrated best in FIG. 8, the overall shape of unit 132 generally conforms to the shape of the capsule. The unit 132 is of sufficient size so that optic combination 136 mildly urges against the posterior wall 60 of the capsule 20, while the anterior portion 138 urges against the anterior wall 52 of the capsule 20.

The embodiment of FIGS. 8-9 has been described as a capsular-shape retaining device having a posterior optic 158 and an anterior opening 142, but it will be appreciated that the unit 132 can be reversed within the eye. That is, it could be positioned to have a posterior opening and an anterior optic. Either way, the unit 132 does not accommodate.

### 7. Embodiment of FIG. 10

Referring to FIG. 10, a capsular shape-restoring device 164 is shown. Device 164 comprises an anterior portion 166, a posterior portion 168, and an equatorial portion 170. Equatorial portion 170 comprises a sidewall 171 that is arcuate in cross-section.

The equatorial portion 170 comprises a plurality of spaced-apart legs 172 radiating therefrom and extending towards the anterior portion 166, and a plurality of spaced-apart legs 174 radiating therefrom and extending towards the posterior portion 168. The legs 172, 174 are similar to those in FIG. 7 in that they are arcuate in cross-section and comprise respective base ends 176, 178 and respective free ends 180, 182. As illustrated, base ends 176, 178 are integrally formed with equatorial portion 170, while free ends 180, 182 terminate without contacting another part of the device 164, thus forming an anterior opening 184 and a posterior opening 186.

As with the previously described embodiments, the overall shape of device 170 generally conforms to the shape of capsule 20.

With each of the devices described above and shown in the figures, an ophthalmic surgeon would remove the natural lens (e.g., during cataract surgery) by conventional methods, leaving an opening 188 in the anterior wall 52 of the capsule 20. The device is then folded into a compact size for insertion into the capsule 20 through the opening 188. Once inserted, the capsule 20 is filled with fluids (e.g., saline solution) which enter the chamber of the device, causing it to return to its original, unfolded state, thus substantially (and more preferably completely) returning the capsule 20 to its natural shape prior to removal of the natural crystalline lens. There is no need to suture the device to the capsule 20 because, due to the size and shape of the inventive device, it will not rotate or shift within the capsule 20.

Regardless of the embodiment used and whether an optic is present, the capsular shape-restoring devices of the present invention do not provide accommodation. Rather, the devices are designed to restore the shape of the capsular bag to its natural state (i.e., its shape when the natural crystalline lens is still present). When an optic is present, improved vision may occur, but the patient would not achieve accommodation from the inventive device alone. Thus, the accommodation achieved with this device alone will be less than about 0.5 diopters, preferably less than about 0.2, and even more preferably about 0 diopters.

This lack of accommodation is the result of two possible factors, depending upon the embodiment. One, if an optic is not present, the device is not affecting eyesight at all, and the device simply restores the capsular bag to its natural state. Two, if an optic is present, the optic will not move (or it will at least be essentially free of moving) axially through the eye, nor will the optic change shape. Furthermore, if an optic is present, the walls of the inventive device are sufficiently rigid that the device will not deform or move in response to ciliary body movement.

Wall rigidity can be achieved by forming the walls from a rigid material (e.g., plastics, rubbers, and mixtures thereof) or, if a less rigid material is selected, rigidity can be achieved by making the device walls thicker. Thus, the device will remain essentially free (and more preferably completely free) of movement when subjected to a radially distributed force of less than about 11 g, preferably less than about 15 g, more preferably less than about 30 g, and even more preferably less than about 45 g. Stated another way, the device will only move when subjected to a radially distributed force that is at least about 11 g, preferably at least about 15 g, more preferably at least about 30 g, and even more preferably at least about 45 g.

It will be appreciated that the inventive device solves the problems in the art in that it substantially restores the capsule to its natural state. As a result, the vitreous is not allowed to move any more than it would if the natural crystalline lens were still present. Furthermore, because the device substantially conforms to the natural shape of the capsule in its natural state, there is a secure fit near the equator of the bag, causing the device to push tightly against the posterior capsule wall and delay, and preferably even prevent, the onset of posterior capsular opacity. Finally, another advantage of the present device is that it can be provided without an optic and used in conjunction with commercially available accommodating lenses.

Although the invention has been described with reference to the preferred embodiment illustrated in the attached drawing figures, it is noted that equivalents may be employed and substitutions made herein without departing from the scope of the invention as recited in the claims. For example, while the foregoing method of inserting the device into the capsule presumed that a portion of the anterior wall of the capsule would be removed with the natural lens, it will be appreciated that it may be possible to insert the device through an incision in the anterior wall. Furthermore, while the foregoing description discloses that the device could be utilized in cataract patients, the device may be used in any situation where the natural lens was removed.

I claim:

1. A capsular, shape-restoring device for implantation substantially within the confines of the capsule of a human eye between the anterior and posterior capsule walls, said device comprising a body presenting a discoid shape that generally conforms to the shape of the capsule, said body comprising:
a posterior face that is configured for yieldable engagement with the posterior capsule wall;
an anterior face that is configured for yieldable engagement with the anterior capsule wall;
a curved sidewall joining the anterior and posterior faces; and
an opening formed in at least one of said posterior face and said anterior face, wherein said device is non-accommodating.

2. The device of claim 1, said body being unitarily formed.

3. The device of claim 1, said device further comprising an optic coupled to said body and positioned opposite the opening.

4. The device of claim 3, wherein said optic is integrally formed with said body.

5. The device of claim 3, wherein said optic is physically distinct from said body.

6. The device of claim 3, wherein said optic is coupled to said posterior face, and said opening is at said anterior face.

7. The device of claim 3, said optic presenting a convex anterior surface and a convex posterior surface.

8. The device of claim 1, said body comprising a material selected from the group consisting of plastics, rubbers, and mixtures thereof.

9. The device of claim 1, wherein said posterior face, said anterior face, and said curved sidewall cooperatively form a chamber within said body.

10. The device of claim 1, said device providing less than about 0.5 diopters of accommodation.

11. The device of claim 10, said device providing about 0 diopters of accommodation.

12. The device of claim 1, wherein said body will remain essentially free of movement when subjected to a radially distributed force of less than about 11 g.

13. The device of claim 1, wherein said curved sidewall comprises a series of spaced-apart openings formed therein.

14. The device of claim 1, wherein said curved sidewall comprises a plurality of legs that are arcuate in cross-section, said legs having respective first and second ends, and the first end of each leg being connected to at least one of said posterior and anterior faces.

15. The device of claim 14, wherein the second end of each leg is free-standing.

16. The device of claim 1, wherein said curved sidewall comprises a plurality of legs that are arcuate in cross-section and that extend from said curved sidewall towards at least one of said posterior and anterior faces.

17. The device of claim 16, wherein said curved sidewall comprises a second plurality of legs that are arcuate in cross-section and that extend from said curved sidewall towards the other of said posterior and anterior faces.

18. A capsular, shape-restoring device for implantation substantially within the confines of the capsule of a human eye between the anterior and posterior capsule walls, said device comprising a body presenting a discoid shape that generally conforms to the shape of the capsule, said body comprising:
a posterior face that is configured for yieldable engagement with the posterior capsule wall;
an anterior face that is configured for yieldable engagement with the anterior capsule wall;
a curved sidewall joining the anterior and posterior faces; and
an opening formed in at least one of said posterior face and said anterior face,
wherein said body will remain essentially free of movement when subjected to a radially distributed force of less than about 11 g.

19. The device of claim 18, said body being unitarily formed.

20. The device of claim 18, said device further comprising an optic coupled to said body and positioned opposite the opening.

21. The device of claim 20, wherein said optic is integrally formed with said body.

22. The device of claim 20, wherein said optic is physically distinct from said body.

23. The device of claim 20, wherein said optic is coupled to said posterior face, and said opening is at said anterior face.

24. The device of claim 20, said optic presenting a convex anterior surface and a convex posterior surface.

25. The device of claim 18, said body comprising a material selected from the group consisting of plastics, rubbers, and mixtures thereof.

26. The device of claim 18, wherein said posterior face, said anterior face, and said curved sidewall cooperatively form a chamber within said body.

27. The device of claim 18, wherein said curved sidewall comprises a series of spaced-apart openings formed therein.

28. The device of claim 18, wherein said curved sidewall comprises a plurality of legs that are arcuate in cross-section, said legs having respective first and second ends, and the first end of each leg being connected to at least one of said posterior and anterior faces.

29. The device of claim 28, wherein the second end of each leg is free-standing.

30. The device of claim 18, wherein said curved sidewall comprises a plurality of legs that are arcuate in cross-section and that extend from said curved sidewall towards at least one of said posterior and anterior faces.

31. The device of claim 30, wherein said curved sidewall comprises a second plurality of legs that are arcuate in cross-section and that extend from said curved sidewall towards the other of said posterior and anterior faces.

32. A capsular, shape-restoring device for implantation substantially within the confines of the capsule of a human eye between the anterior and posterior capsule walls, said device comprising a body presenting a discoid shape that generally conforms to the shape of the capsule, said body comprising:
a posterior face that is configured for yieldable engagement with the posterior capsule wall;
an anterior face that is configured for yieldable engagement with the anterior capsule wall;
a curved sidewall joining the anterior and posterior faces; and
an opening formed in at least one of said posterior face and said anterior face,
wherein said device does not include an optic.

33. The device of claim 32, wherein said body comprises an opening at both of said posterior and anterior faces.

34. The device of claim 32, said body being unitarily formed.

35. The device of claim 32, said body comprising a material selected from the group consisting of plastics, rubbers, and mixtures thereof.

36. The device of claim 32, wherein said posterior face, said anterior face, and said curved sidewall cooperatively form a chamber within said body.

37. The device of claim 32, wherein said body will remain essentially free of movement when subjected to a radially distributed force of less than about 11 g.

38. The device of claim 32, wherein said curved sidewall comprises a series of spaced-apart openings formed therein.

39. The device of claim 32, wherein said curved sidewall comprises a plurality of legs that are arcuate in cross-section, said legs having respective first and second ends, and the first end of each leg being connected to at least one of said posterior and anterior faces.

40. The device of claim 39, wherein the second end of each leg is free-standing.

41. The device of claim 32, wherein said curved sidewall comprises a plurality of legs that are arcuate in cross-section and that extend from said curved sidewall towards at least one of said posterior and anterior faces.

42. The device of claim 41, wherein said curved sidewall comprises a second plurality of legs that are arcuate in cross-section and that extend from said curved sidewall towards the other of said posterior and anterior faces.

43. A method of restoring the natural shape of a capsular bag, said method comprising the steps of:
providing a capsular bag whose natural crystalline lens has been removed;
inserting a non-accommodating device into said capsular bag, said capsular bag being substantially returned to its natural shape after said inserting step.

44. The method of claim 43, wherein said device does not include an optic.

45. The method of claim 43, wherein said device comprises:
a posterior face that is configured for yieldable engagement with the posterior capsule wall;
an anterior face that is configured for yieldable engagement with the anterior capsule wall;
a curved sidewall joining the anterior and posterior faces; and
an opening formed in at least one of said posterior face and said anterior face.

46. The method of claim 43, wherein said body will remain essentially free of movement when subjected to a radially distributed force of less than about 11 g.
